(19) United States
(12) Patent Application Publication
(10) Pub. No.: US 2022/0369390 A1
(43) Pub. Date: Nov. 17, 2022

(54) Method and System to Pair an Article to a User

(71) Applicant: Lampros Kourtis, Larisa (GR)

(72) Inventor: Lampros Kourtis, Larisa (GR)

(21) Appl. No.: 17/436,446

(22) PCT Filed: Mar. 3, 2020
(86) PCT No.: PCT/IB2020/051771

§ 371 Date: Sep. 3, 2021

Related U.S. Application Data
(60) Provisional application No. 62/813,264, filed on Mar. 4, 2019.

(51) Int. Cl. H04W 76/10; A61B 5/00; A61B 5/11

(52) U.S. Cl. CPC H04W 76/10; A61B 5/6801; A61B 5/11

(57) ABSTRACT

A method and system to seamlessly pair a user to an article includes matching their respective inertial profile. The system comprises of a user sensor that can capture and communicate the motion profile of the user or a part of the user as well as an article sensor that can capture and communicate the motion profile of the article. Both motion profiles are communicated to a pattern matching module. When the article and the user are spatially interacting for at least a minimum period of time, the pattern matching module can determine the level of similarity between the respective profiles. A decision to pair the article to the user is produced based on said level of said profiles similarity.

### CROSS-REFERENCE TO RELATED APPLICATIONS

This patent application is a U.S. national phase patent application of International Patent Application No. PCT/IB2020/051771, filed Mar. 3, 2020, which is fully incorporated herein by reference for all purposes.

### FIELD OF THE INVENTION

The present invention relates generally to the field of human-machine interfaces and more particularly to the field of Internet of Things. The invention refers to a method to create a link between articles and users or between articles or between users based on the similarity of their motion profile, once they interact.

### BACKGROUND OF THE DISCLOSURE

For the purpose of this invention disclosure, “pairing” refers to the action of linking or assigning one or a plurality of articles to a user, or to other articles, or a user to a user, to allow further communications or actions between them. “Article” is defined a particular object or item that is intended to interact with a user. “User” is a person who uses or operates something, and in this case, an article. “Inertial profile” refers to the properties of motion as a user or an article, or parts of them, move in space.

In the internet of things (IoT) world, it is imperative to know that a particular user is using an article so that proper actions take place. Pairing of an article to a user is a process that sometimes requires user effort (such as scanning a QR code, or performing Bluetooth Pairing), suggesting that wide, seamless, applications in the internet of things may be hindered. Pairing currently is performed using technologies such as Bluetooth, Wi-Fi, NFC, Optical, by QR or bar code scan and others. Examples of pairing are quite common, such as the link between a Bluetooth headset and a user's mobile phone, or a user's magnetic card and a security system reader, or an RFID tag attached to a book and a RFID reader attached to a user's wearable device, or a QR code on a medical device and a user's phone QR code reader. Most of the aforementioned methods interrupt the user experience between the user and the article introducing a separate authorization/pairing sequence before operating the article.

In a lot of these cases, pairing is required to authorize use of an article. In order to enable the user to use a particular service, the article needs to know that said user has permission to employ such service. One example is in ride sharing applications, such as electric scooters or bicycles, where in order to authorize use of the vehicle, the user riding the vehicle needs to have the right credentials, based on the user's account properties (payment, consent etc.). Another example is in a manufacturing area, where an operator of a piece of equipment needs to have the right access to said piece of equipment based on function, training and quality control. Another example is in the medical device field, where a user needs to deliver a treatment (device or drug) to himself or to somebody else, and where the operation of such device or drug needs to be enabled and accounted to the particular user, for treatment adherence purposes. Another example is in the case where a smart device (such as a smartphone) needs to be unlocked when a user picks it up. Another example is in the exercise and rehabilitation field, where a user's performance is measured by identifying which piece of exercise equipment, he/she is using.

In all of the abovementioned examples, the authorization/pairing process occurs while the user is interacting with the device and hence does not interfere with the user experience. Moreover, the user and the article share, at least in part, a common inertial profile. For example, a drug delivery device, that is used by a patient, will at some point during its use, have a similar inertial profile with the patient's hand. In another example, a dumbbell or another piece of rehabilitation equipment used by a person, will at some point during its use, have a similar inertial profile with the patient's hand. In another example, an electric scooter used by a person, will at some point during its use, have a similar inertial profile with the user's body.

In all of these examples, and for different purposes, there is a need to create a link between the article and the user.

### SUMMARY

The invention discloses a method and a system to seamlessly pair a user to an article by matching their respective inertial profile. The system comprises of a user sensor that can capture and communicate the motion profile of the user or a part of the user as well as an article sensor that can capture and communicate the motion profile of the article. Both motion profiles are communicated to a pattern matching module. When the article and the user are spatially interacting for at least a minimum period of time, the pattern matching module can determine the level of similarity between the respective profiles. A decision to pair the article to the user is produced based on said level of said profiles similarity.

### BRIEF DESCRIPTION OF THE DRAWINGS

FIG. 1 depicts a schematic summary of the system comprising an article (2) that carries an article sensor (1) and that has an inertial moment unit (3), a processing unit (4) and a communications unit (5). FIG. 1 also depicts a user (7) that carries a wearable device (6) that in turn comprises an inertial moment unit (8), a processing unit (9) and a communications unit (10). The signals from both the article and the user communication units (5 and 10 respectively) transmit signals between them or between each of them and a computerized pattern matching module (11) that in turn is connected to a decision making module (12) capable of making a decision and transmitting it back to the user and/or the article. The pattern matching module (11) compares the motion profile of the article (15) to the motion profile of the user (16), to determine if the two of them are spatially interacting.

FIG. 2 depicts a user (7) holding a user sensor (6), that is about to operate an article, in this case, a piece of exercise equipment (14) that holds an article sensor (1). Furthermore, the user sensor (6) is a wearable or smartwatch and the piece of exercise equipment (14) is a dumbbell. Once the user (7) interacts spatially with the dumbbell (14), their motion profiles are similar and linking of the user to the dumbbell can occur for monitoring and training purposes.

FIG. 3 depicts a user (7) that holds a user sensor (6). The user (7) is about to ride on an article, in this case, a vehicle (13) that holds an article sensor (1). Furthermore, the vehicle (13) is an electric scooter. Once the user (7) interacts spatially with the vehicle (13), their motion profiles are similar, and authorization of the ride can be determined.

FIG. 4 depicts two simultaneous signals (15 and 16) that come from a) the article IMU (3) and b) the user IMU (8) respectively in a period where the user (7) and the article (2) happen to spatially interact with each other. The two signals (15 and 16) present portions (17 and 18 respectively) where the similarity of the signals is high. These portions are identified by the pattern matching module that determines if the user (7) and the article (2) are linked. In this figure, the accelerometer L2 norm is presented, however, other motion metrics can be included.

FIG. 5 depicts a general flowchart of a ride share authorization process. It starts with the user motion profile (16) and the article motion profile (15). For each, motion profile features are extracted and then those features are compared for similarity. If the similarity is high enough, pairing occurs between the user and the vehicle and a decision-making module can check if the user has the credentials to use the vehicle and performs the billing if needed. The decision is then communicated back to the vehicle that in turn unlocks for use.

FIG. 6 depicts a general flowchart of the pairing action starting with the user motion profile (16) and the article motion profile (15). For each, motion profile features are extracted and then those features are compared for similarity. If the similarity is high enough, pairing occurs between the user and the article and further actions can occur.

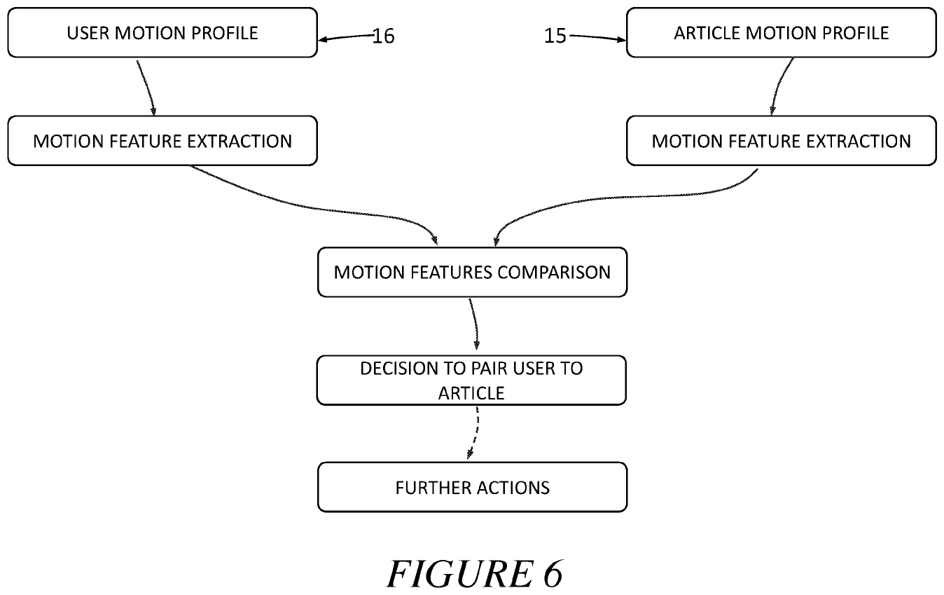

FIG. 7 depicts a flowchart of the pairing action, between multiple articles paired to a user as in the case of using a piece of sports or rehabilitation equipment that holds multiple weights (stacked weights). For each of the user and the articles, motion profile features are extracted and then those features are compared for similarity. If the similarity is high enough, pairing occurs between the user and the articles that hold said high similarity, further actions can occur, such as determining that the user is lifting a given weight that is related to the sum of the weights of the articles (stacked weights) that are paired.

FIG. 8 depicts a user (7) and a piece of exercise equipment (19) namely a latissimus dorsi pulldown machine (lat pulldown). The machine holds multiple stacked weights (2 and 2′) that each hold an article sensor (1 and 1′). Depending on the user selection, a number of the stacked weights are lifted, in this case weights A, B, C, D, E (cumulatively, 2), whereas weights F and G (cumulatively 2′) are not lifted. The motion profile (15) of weights A, B, C, D, E is shown on the upper right corner whereas the motion profile (15) of the weights F, G is shown on the lower right corner. The user (7) is pulling the bar (23) and the wearable sensor (6) is registering the motion profile (16) that matches that of weights A, B, C, D, E. As such, the total weight that the user is lifting is related to the sum of weights A, B, C, D, E.

FIG. 9 depicts a flowchart of a general pairing strategy that includes geolocation and Bluetooth proximity. First, the location of the user and the article is examined to determine if the user and the article are nearby. This can be enabled using GPS or other geo-positioning technologies with their location being shared on a common server that performs the proximity test. If the article and the user are nearby, the user's Bluetooth module is scanning for article's Bluetooth module advertising. If the Bluetooth modules are in range, then the article motion profile (15) and the user motion profile (16) are compared for similarity. If the two profiles are similar enough, a decision is made to pair article to the user.

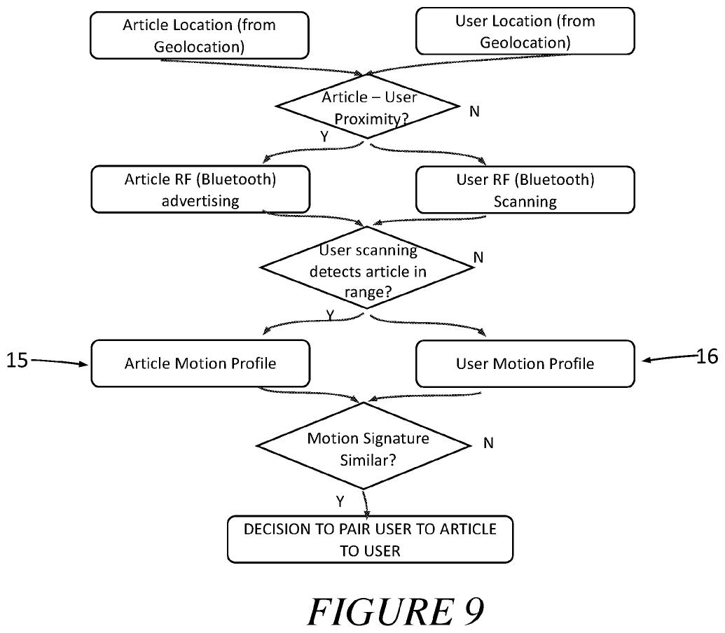

FIG. 10 shows a time-frequency plot of the motion profile coming from several articles during a series of activities that the user is engaged: Subplot 10A shows spectrogram of the resultant (L2 norm of the three components) acceleration profile of a smartwatch worn on a user while performing various activities. Subplot 10B shows the resultant acceleration profile of a sensor attached to the frame of a city bike from a ridesharing service. Subplot 10C shows the resultant acceleration profile of a sensor attached to the handle of an elliptical machine. Subplot 10D shows the resultant acceleration profile of a sensor attached to a dumbbell.

FIG. 11 is a closeup of a piece of exercise equipment (19) showing the stacked weights (2), each of which has a sensor (1) attached to it, each sensor having a communication unit that allows communication between them. Communication can be optical via LED (20) and photodiode (21), acoustic via speaker and microphone, or RF via electromagnetic transceivers (5). This embodiment allows for intra-article communication to reduce the burden on the Bluetooth sensors.

FIG. 12 depicts a method to unlock a smartphone using inertial profile matching. A user (7) is picking up a smartphone (22) in order to use it. The user (7) is wearing a smartwatch (6), equipped with an IMU. When the motion profile (16) of the user's hand, as recorded by the smartwatch, matches the motion profile (15) of the smartphone, the smartphone is unlocked so that the user can use it.

FIG. 13 depicts a piece of exercise equipment, namely a weight bar (23) and weight plates (2), each equipped with an article sensor (1). When the user (7) lifts said bar (23), the motion profile (16) of his/her wearable (6) matches the motion profile (15) of the bar (23) as well as the motion profile (15) of the weight plates (2). The pattern matching module determined if the similarity between the profiles (15 and 16) is high enough to register this activity to the user (7). The rest of the available weight plates (2′) that are equipped with article sensors (1′) present a motion profile (15′) that is different from the user's motion profile (16) and as such are not paired with the user (7).

FIG. 14 depicts an embodiment of the invention where the article sensor (1) is part of a medical device such as an autoinjector (24). The user (7) handles the autoinjector (24) prior to injecting the drug, while his wearable (6) captures the motion profile during that operation. The two motion profiles, from the article (24) and the user (7) present similarities at some point of said operation, hence the pattern matching module and the decision making modules can determine if and how the user is using the article (7) and report on medication adherence to their doctor or payer.

FIG. 15 depicts an embodiment of the invention where the article is a static piece of sports equipment, namely a yoga mat (25) that comprises a piezoresistive element (26) that captures the force profile (27) on its surface. The user (7) is performing an exercise while the wearable (6) is capturing the user's motion profile (16). The pattern matching in this case compares features from the user motion profile (16) to features from the article's force profile (27).

FIG. 16 depicts a flowchart of a user-article authorization or registration procedure using two party system (without the need for a server-side party). The article sensor detects motion using an IMU metric threshold, and if motion is detected, the motion profile is encoded and featurized. The sensor's BTLE advertising string adjusts based on the motion profile. At the same time, a user sensor is scanning available BTLE, and if one that has a known serial string is found, a temporal (trial) pairing request is sent. The user sensor is extracting motion profile features and if temporal pairing is successful, it transmits said features to the article's sensor. The article sensor performs a comparison of the motion profile features coming from the article and the user sensors. If the level of similarity is high enough, article authorization occurs. If not, an alert may be issued or the temporal pairing is dropped.

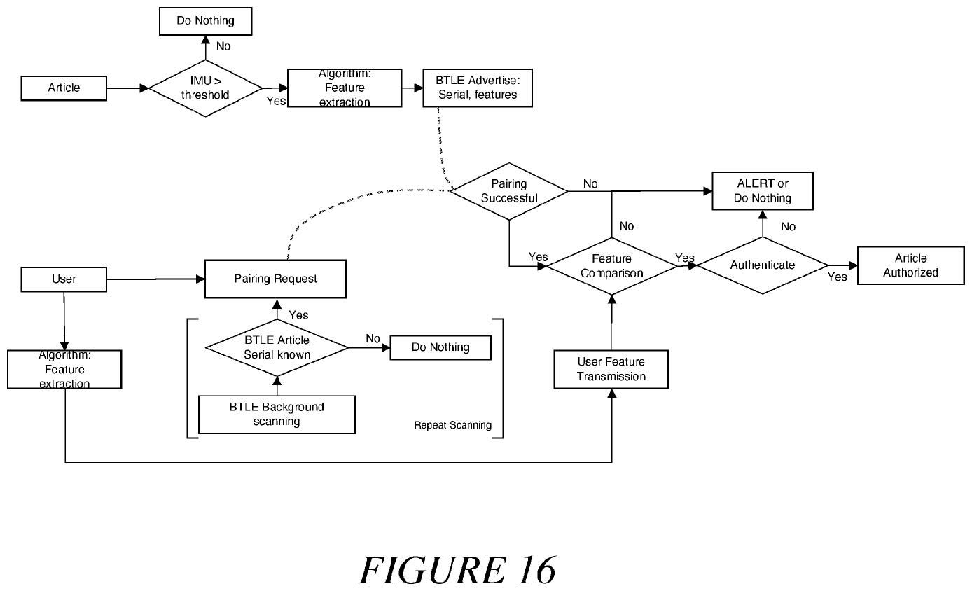

### DETAILED DESCRIPTION

The Present Invention Includes the Following Components:

A) an Article Sensor (1) that is Attached to an Article (2) Comprising
a. An Inertia Moment Unit—IMU (3) or other Sensing Unit
b. A Processing Unit (4)
c. A Communication Unit (5)

The Processing Unit (4) calculates features coming from the IMU Signals (15) and then the Communication Unit (5) transmits said features to an on-site or off-site Computerized Pattern Matching Module (11).

B) A User Sensor (6) that is Attached to a User (7) or Another Article Comprising
a. An IMU (8)
b. A Processing Unit (9)
c. A Communication Unit (10)

The Processing Unit (9) extracts features from the IMU Signals (16) and then the Communication Unit (10) transmits said features to an on-site or off-site Computerized Pattern Matching Module (11).

C) An on-site or off-site receiving and Computerized Pattern Matching Module (11) that can be either remote (server location) or it can comprise a component of A) or a component of B).

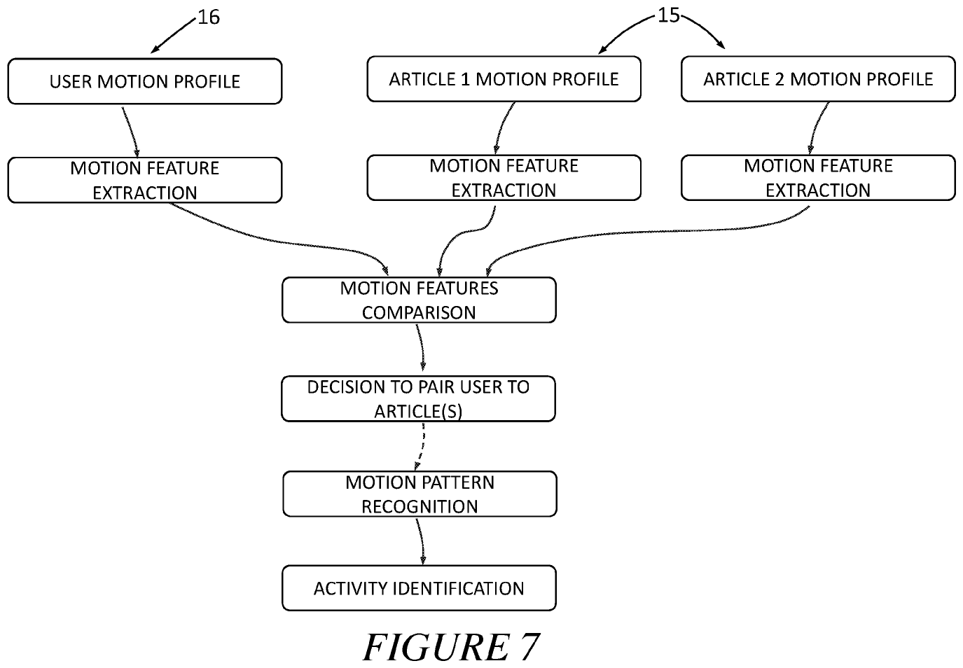

The Computerized Pattern Matching Module receives the signals or features (15, 16) provided by the Article Sensor (1) and the User Sensor (6) and performs pattern matching operations to determine feature similarity. Depending on the similarity level, the Article (2) is paired to the User (7) so that further exchange of information can occur. The pairing decision can be communicated to a Decision-Making Module (12), such as a server. FIG. 7 shows a logical diagram of the pairing operation.

The present invention discloses a method to seamlessly pair a plurality of articles (2 or 14 or 13 or 20) to a user (7) by comparing their respective motion patterns and assessing similarity in said patterns. These patterns are recorded using Inertia Moment Units (IMU) that may include a 1, 2, or 3 axes accelerometer, a 1, 2, or 3 axes gyroscope, a 1, 2, or 3 axes magnetometer and a 1 axis altimeter.

The present invention relates the IMU Signal (15) of an Article Sensor (1) that is attached to an Article (2) to the IMU Signal (16) of a User Sensor (16), worn by or attached to the user (7). Once the User (7) holds, operates, rides, steps, or otherwise spatially interacts with the Article (2), the motion patterns of their two respective IMUs present matching features. If the degree of matching between the patterns or features is high enough, then a Computerized Pattern Matching Module (11) that receives the respective signals/patterns or features, can determine that a particular User (7) is interacting with one or a plurality of Articles (2) and thus link the two so that further exchange of information can occur.

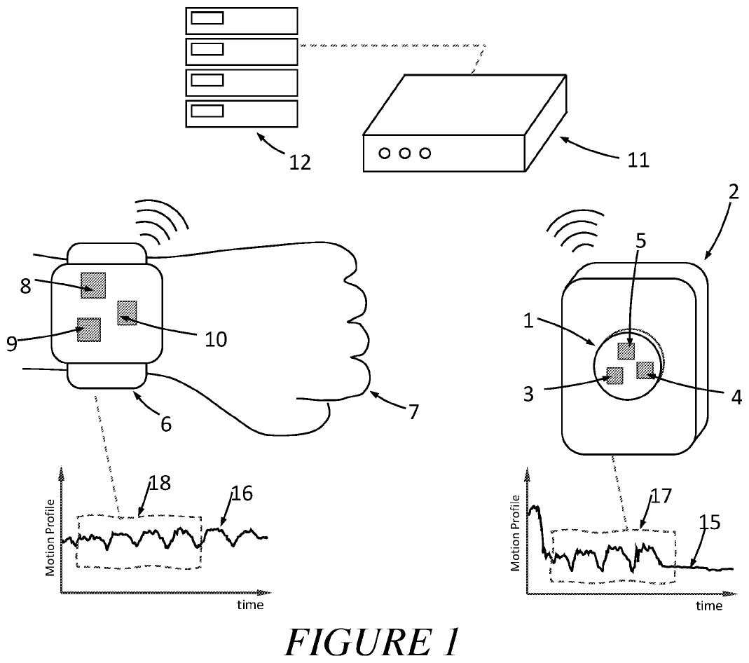

In one embodiment shown in FIG. 1, the article IMU signal (15) or features extracted from it is/are transmitted to a computing unit (11) via Bluetooth, GSM, wifi, or ultrasound method. The wearable IMU signal (16) or features extracted from it is/are transmitted to a computing unit (11) found on a server via Bluetooth, cellular network communication such as 2G/3G/4G/5G or similar, wifi, UWB, optical, or ultrasound communication method. The computing unit (11) compares the two signals (15, 16) or signal features and identifies matching patterns (17 and 18). If that matching pattern is strong enough, the article (2) is paired to the wearable (6) and in turn to the user (7).

In one embodiment, features extracted from the article IMU signal are transmitted by means of Bluetooth advertising or ultrasound or wifi or optical or other RF advertising such as Ultra-Wideband (UWB). The wearable device picks up the Bluetooth or ultrasound or optical or wifi or other RF advertising using the Bluetooth module or microphone or wifi or another RF sensor respectively. The features are then compared to the features extracted from the wearable IMU, and if that matching pattern is strong enough, the article is paired to the wearable and in turn to the user.

In one embodiment, the article sensor (1) transmits IMU features in combination with features derived from the baseband signal of the broadband communication module such as Bluetooth, wifi or Ultra-wideband. Similarly, the wearable (6) is also producing IMU features and features derived from the baseband signal of the communication module. Baseband signals include but are not limited to time of flight (ToF), phase shift, broadband signal strength, Received Signal Strength Indicator (RSSI) etc. By combining IMU features with wireless communication features the specificity of the pairing algorithm is improved.

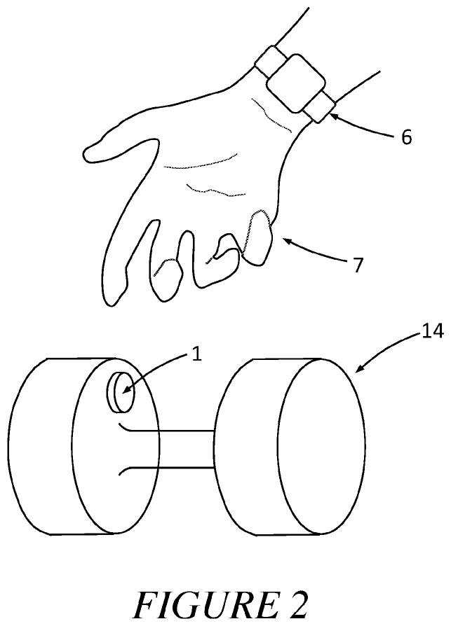

One embodiment of the invention has an application in sports and more particularly, gym equipment as shown in FIG. 2. When a user (7) carrying a wearable (6), for example watch, holds a particular piece of gym equipment (14) that has an IMU sensor (6) attached to it, their IMU signal features match, hence the user (7) and the piece of gym equipment (14) are paired. Alternatively, when a user steps or operates another piece of equipment, the IMU signal profiles match, hence the user and the piece of equipment are paired. Each piece of equipment may hold a digital label, i.e. dumbbell, or treadmill, or free weight, or a weights bar, or elliptical machine, or a step machine, or a roman chair, or an abs chair, or any adjustable stack weight machine. An IMU sensor can be placed in each of the stacked weights, and their signal profiles can in turn be matched. This way, not only a user is paired to a piece of equipment but, the actual weight lifted can be calculated by summing the weights of the paired stacked weights.

In one embodiment, the present invention can be used to pair a user to a dumbbell (14) and determine the actual weight, number of repetitions, and the motion profile. A sensor (6) is attached to a dumbbell (14). The IMU signal (15) or features extracted from it are transmitted to a computing unit (11) using a Bluetooth hub. The wearable IMU signal (16) or features extracted from it are also transmitted to a computing unit (11) using a wifi or 2G/3G/4G/5G hub. The computing unit (11) compares the signals (15, 16) from the entire fleet of dumbbell sensors and other pieces of gym equipment and matches the user wearable IMU signal profiles to the dumbbell IMU signal profiles. When a matching pattern is strong enough, the dumbbell and the user are paired. Geolocation information, such as GPS or GLONASS, can be factored in to narrow the search between the entire fleet of articles across the world. Alternatively, the dumbbell IMU signal or features extracted from it are encoded and advertised using the Bluetooth advertising label or using an encoded ultrasound signal or using an optical signal. The wearable picks up the advertising using the Bluetooth or the microphone or the optical signal respectively. For example, the ultrasound signal can be a direct conversion of the acceleration of an article into ultrasound frequency or ultrasound intensity. More specifically, the frequency of the emitting ultrasound can be proportional to the acceleration of the article as recorded by the IMU (3). Likewise, the optical signal can be a direct conversion of the acceleration of an article into optical frequency or intensity. More specifically, the optical signal intensity or wavelength can be proportional to the acceleration of the article as recorded by the IMU (3). The computerized pattern matching unit (11) calculates a features match score, and if the score is high enough, the dumbbell (14) and the user (7) are paired. Such a score can be a softmax function for example, or the squared difference between the two curves. The dumbbell sensor (1) holds a digital description containing information such as the actual weight in kg, the type of weight etc. The sensor (1) may be attached to the dumbbell (14) using an adhesive tape or, other mechanical means. Once the dumbbell (14) and the user (7) are paired, the system can record the number of repetitions by counting the repeated pattern count, as well as the actual weight lifted. It can also provide other trajectory specific information, to assess the exact type of the activity (for example, biceps pull vs triceps push exercise). This identification can be done by using Machine Learning techniques, by accumulating tagged motion patterns from a plurality of users and then obtaining a score that relates the profile to a particular exercise type. This example refers to dumbbell pairing, but it can also apply to all other pieces of gym equipment.

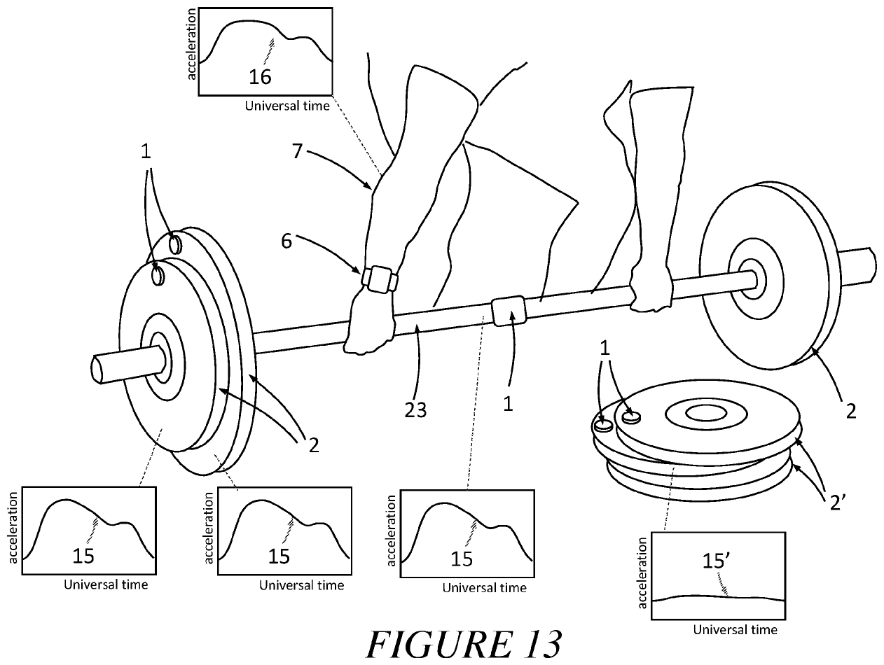

In one embodiment shown in FIG. 13, the present invention can be used to pair a user to a number of weight plates (2) and a bar (23) to a user (7), and determine the actual weight, number of repetitions, and the motion profile of the activity. The IMU signal (15) or features extracted from the plurality of article sensors (1) located on each weight plate (2) and the bar are transmitted to a computing unit (11) using a Bluetooth hub. The wearable IMU signal (16) or features extracted from it are also transmitted to a computing unit (11) using a wifi or 2G/3G/4G/5G hub. The computing unit (11) compares the signals from the entire fleet of sensors and other pieces of gym equipment and matches user wearable IMU signal profiles to the bar and weight plates IMU signal profiles. When a matching pattern is strong enough, the bar (23) and weight plates (2) and the user (7) are paired. Geolocation information can be factored in to narrow the search between the entire fleet of articles across the world. Each article sensor (1) holds a digital description containing information such as the type of equipment, i.e. bar vs weight plates vs stacked weight vs other exercise equipment, the actual weight of each weight plate in kg, the type of weight etc. The article sensor (1) may be attached to the weight plates (2) or the bar (23) using an adhesive tape or, other mechanical means. Once the bar (23) and weight plates (2) and the user (7) are paired, the system can record the number of repetitions by counting the repeated pattern number, as well as the actual total weight lifted. It can also provide other trajectory specific information, to assess the exact type of the activity (for example, chest press vs shoulder push). This identification can be done by using Machine Learning techniques, by accumulating tagged motion patterns from a plurality of users and then obtaining a score that relates the profile to a particular exercise type. This example refers to barbell pairing and exercise measuring, but it can also apply to all other pieces of gym equipment.

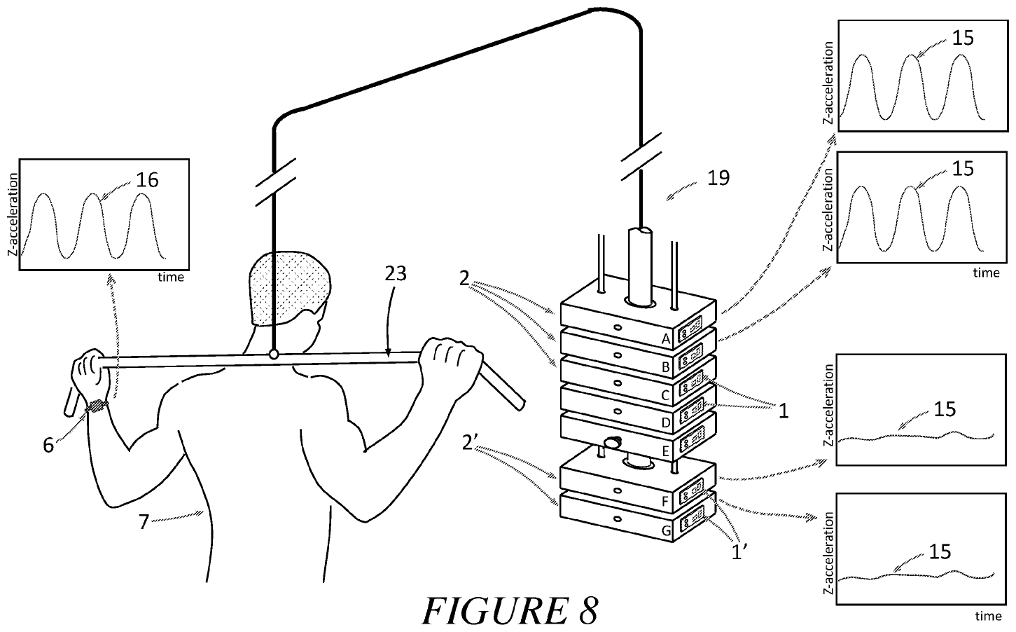

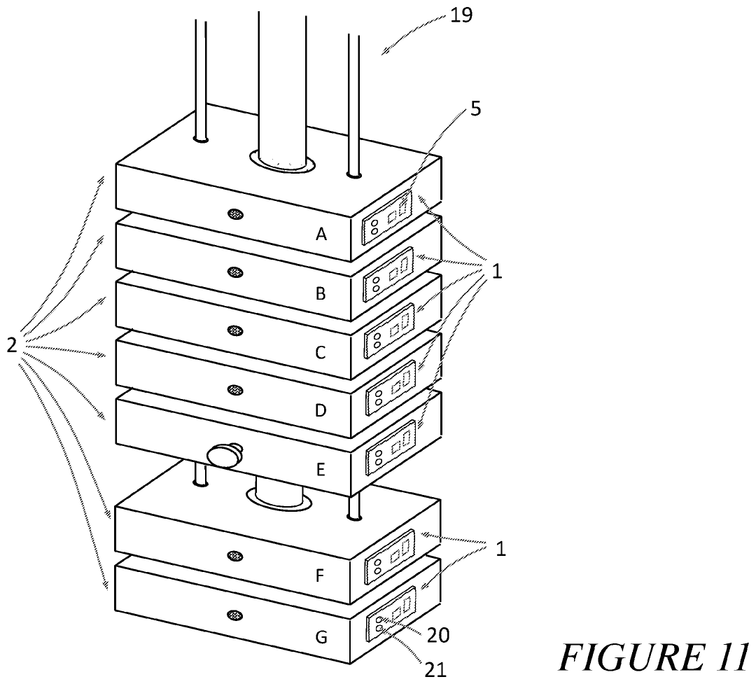

In one embodiment, the present invention can be used to pair a user (7) to a number of stacked weights (2 and 2′) in an exercise machine (19), and determine the actual weight, number of repetitions, and the motion profile of the activity as shown in FIGS. 8 and 11. The IMU signal (15) or features extracted from the plurality of sensors located on each weight plate and the bar are transmitted to a computing unit (11) using a Bluetooth hub. The wearable IMU signal (16) or features extracted from it are also transmitted to a computing unit (11) using a wifi or 2G/3G/4G/5G hub. The computing unit (11) compares the signals from the entire fleet of sensors and other pieces of gym equipment and matches the user wearable IMU signal profiles (16) to the stacked weight plates IMU signal profiles (15). When a matching pattern is strong enough, the stacked weights and the user are paired. Each sensor (1 and 1′) holds a digital description containing information such as the type of equipment, i.e. stacked weight, the actual weight of each weight in kg, the type of weight etc. By adding the weight of each stacked weight (1) that have a high motion profile similarity to motion profile of the user (7), the system can pair these weights (2) but not the rest of them (2′) and thus determine the exact weight that was lifted by the user. In one embodiment, the stacked weights can be paired to each other using a similar procedure. Inter-weight pairing can also happen by optical means: for example, the top stacked weight can communicate to the web via Bluetooth (BT) or wifi connection, but the rest of the weights can communicate with the top stacked weight using LED or ultrasound signaling. In such an embodiment shown in FIG. 11, the sensor that is attached to the top stacked weight (A) is equipped with a photodiode (21) that can pick up the signal from an LED (20) that is placed on a sensor (1 and 1′) that belongs to the next stacked weight (B). The LED (20) signals that the corresponding stacked weight is moving, based on its motion profile. Similarly, the rest of the sensors daisy chain to each other (E to D, D to C, C to B), by signaling each other when they are in motion. Each LED may signal at a different frequency so that the signals can be received in order. In the example shown in FIGS. 8 and 11, only LEDs belonging to sensors attached to stacked weights A, B, C, D, E are on, while the rest (F, G) are off, since they are not moving. The total weight lifted by the user is the sum of weights A+B+C+D+E. The sensor that is attached to the top stacked weight aggregates the information and has a wireless communication module (5) that communicates the information to the cloud. Geolocation information can be factored in to narrow the search between the entire fleet of articles across the world. The sensor device may be attached to the stacked weight plates (2 and 2′) or the bar (23) using an adhesive tape or, other mechanical means. Once the bar and weight plates (2) and the user (7) are paired, the system can record the number of repetitions by counting the repeated pattern number, as well as the actual total weight lifted. It can also provide other trajectory specific information, to assess the exact type of the activity (for example, chest press vs shoulder push). This identification can be done by using Machine Learning techniques, by accumulating tagged motion patterns from a plurality of users and then obtaining a softmax that relates the profile to a particular exercise type.

The present invention can be applied in gym equipment that is static, by picking up slight inertial perturbations that are caused by the movement of a user. For example, a roman chair is a static piece of equipment, yet the elasticity of the materials that comprise it as well as the foundation of the piece of equipment may allow for some splay or micromotion. The inertial pattern of this micromotion may be used to extract features that can be correlated to the inertial pattern of the wearable to uniquely pair the roman chair to the user. The present invention suggests not only article-user pairing but also can help identify and quantify the training. For example, by counting the repeated patterns in a particular interaction, the number of repetitions can be defined. The same principle holds for static abs.

In one embodiment, the article includes a strain gauge assembly or a load cell assembly that measures changes of the normal or shear stresses or combination thereof in one or multiple axis. The changes are recorded by an analog to digital converter equipped by a signal conditioning unit and processed by a processing unit. The article can carry multiple strain gauge assemblies or load cells. The processing unit extracts features from the acquired waveforms and communicates them to the computing unit using a communication module. The computing unit matches the stress distribution profile of the article to the motion profile of the wearable device that is carried by the user. This embodiment is particularly useful in application where there are limited or no moving parts such as when operating a machine. In that case, the motion profile of the user is matched to strain or force profile of the article. In another embodiment, the article can include an IMU and a set of strain gauge or load cell assemblies.

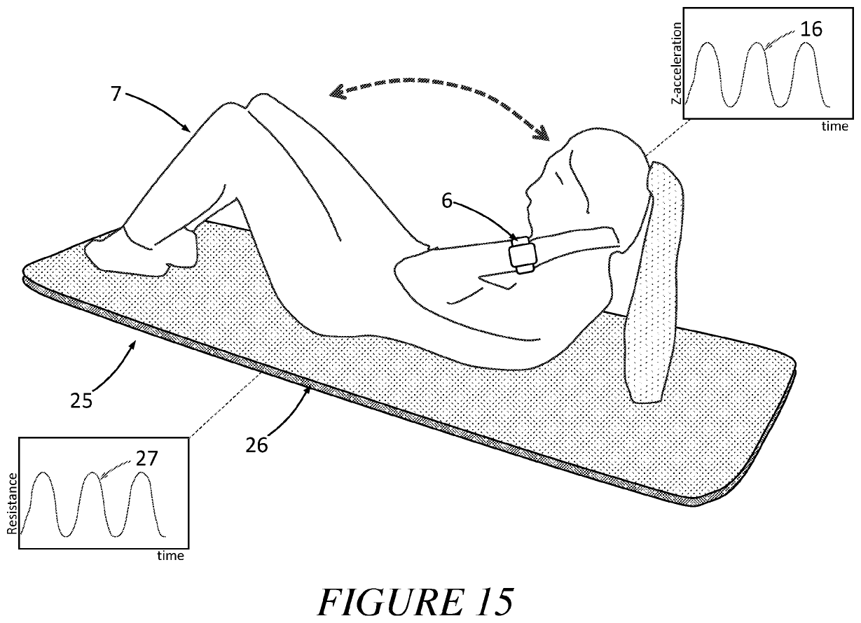

In one embodiment shown in FIG. 15, the article is a static piece of equipment such as a yoga mat (25) that holds a piezoresistive element (26) that is capable of measuring the force profile (25) applied to its surface. The user (7) is performing an exercise that has a motion profile (16). The motion profile (16) is captured by the wearable (7) and more specifically by its IMU (3). The wearable motion profile (16) and the article's force profile (25) are compared to each other to determine similarities, even if the two signals come from different sources (inertia, force).

Generally, the user's motion profile features are compared to another signal profile from an article sensor that measures force or strain or voltage or capacity or resistance. This embodiment can be implemented for example in cases where the user's inertial change does not cause a significant inertial change to the article, but does cause a force.

The present invention can be used to pair a user to a treadmill and assess the number of steps as well as the incline of the treadmill. An IMU sensor is placed on the treadmill. When a user steps on the treadmill and starts running or walking, the user's wearable inertial pattern will provide features that can be correlated to the treadmill (article) inertial profile based on the periodic vibrations that are caused by every step of the user. Such features could be the exact universal time of peak acceleration that is caused by every step, or the frequency and phase of this action. In addition, the incline of the treadmill can be estimated by taking the relative gravity vector component angle as estimated by the multi axis accelerometer in the IMU. This means that once a user is paired to the treadmill, the number of steps, and the incline of the treadmill can be estimated.

The present invention can be used to pair a user to an elliptical machine and assess the number of steps. An IMU sensor is placed on the handle and/or the pedal of the treadmill. When a user steps on the elliptical and starts exercising, the wearable inertial pattern will provide features that can be correlated to the article inertial pattern. Such features could be the exact universal time of peak acceleration that is caused by every step, or the frequency and phase of this action. In addition, the shape of the motion profile can be estimated by processing the IMU data by means of Kalman filtration and subsequent integrations. This allows for a more accurate representation of the actual movement pattern. This means that once a user is paired to the elliptical, the number of steps, and the span of the motion can be estimated.

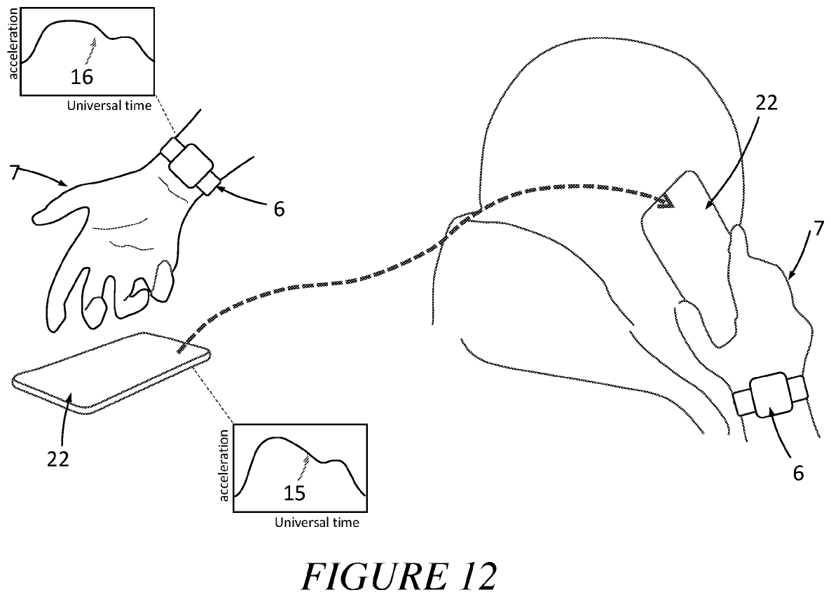

In one embodiment shown in FIG. 12, the present invention can be used in the process of unlocking of a smart device such as a smartphone (22). For privacy and security reasons, smartphones are programmed to lock their screen after a short period of inactivity. Currently, various ways have been identified to facilitate smartphone unlocking, such as the use of a passkey or the use of a face scan or the use of a fingerprint. The present invention discloses a method to unlock the smartphone (22) upon matching of the phone's motion pattern (15) to the motion profile (16) of a wearable (6) that is worn on the hand of a user (7) that is attempting to unlock the smartphone. In this particular case, the wearable and the smartphone may already be pre-paired via Bluetooth, hence the present invention discloses methods of unlocking rather than pairing. The communication between the wearable and the phone may be Bluetooth or ultrasound or other RF. As an example, the phone (22) may emit an ultrasound that encodes the motion profile (15), and the wearable (6) may receive said ultrasound and real time compare the motion profile with its own motion profile (16). The encoding may be such that the intensity of the frequency of the ultrasound emitted by the phone is proportional to the amplitude of the L2 norm of its acceleration. If the level of similarity is high enough, the phone is unlocked without further action. As another example, the phone (22) and the wearable (6) may be pre-paired via Bluetooth, and the motion profile of one may constantly be communicated to the other via the Bluetooth protocol. If the level of similarity between the phone motion profile (15) and the wearable motion profile (16) is high enough, the phone is unlocked without further action.

In one embodiment, the IMU profiles (15, 16) can further be evaluated according to biomechanical aspects, to maximize exercise efficiency and outcomes, for example muscle mass and joint anatomy or potential injuries. An idealized inertial profile may be used as a template and the actual inertial profile form a particular user can be compared to identify areas of improvement. For example, too slow, or too fast. A personal trainer can inspect trajectories and make recommendations. In addition, in rehabilitation centers, this method can help practitioners evaluate the condition of a patient and suggest training programs or determine when they are ready to go back in action.

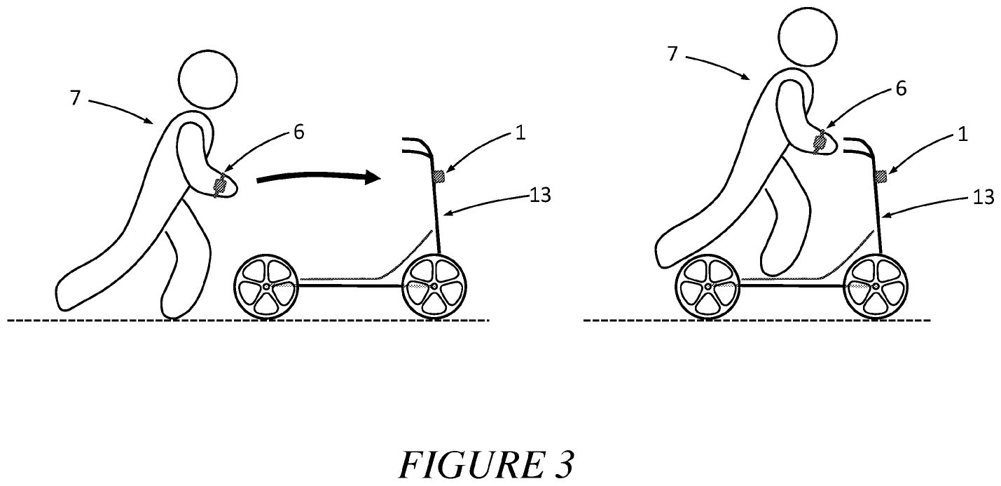

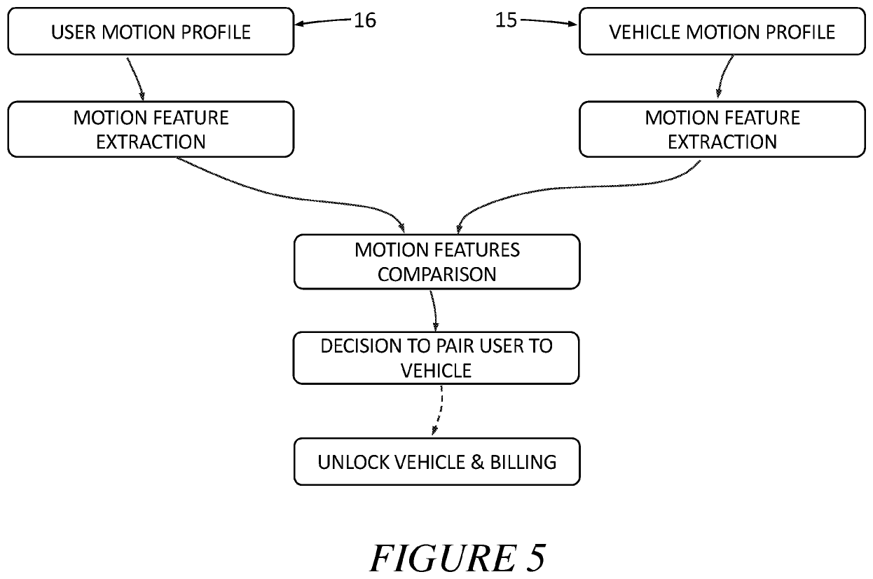

Another embodiment of the invention has an application in ride sharing technologies, for example bicycle, electric bicycle, scooter, electric scooter (13) shown in FIG. 3. For example, when a user (7) that carries a wearable (6) equipped with an IMU (8) rides an electric scooter (13) equipped with a sensor (2) that comprises an IMU (3), the two IMU signal patterns (15, 16) have a high level of similarity. Hence the scooter (13) is paired to the wearable (6) and therefore to the user (7) so that further actions can take place. The scooter IMU signal (15) or features extracted from it are transmitted to a computing unit (11). The wearable IMU signal (16) or features extracted from it are also transmitted to a computing unit (11). The computing unit (11) compares the signals from the entire fleet of scooters and entire user population and matches the user wearable IMU signal profiles to scooter IMU signal profiles as shown in FIG. 5. When a matching pattern is strong enough, the scooter (13) and the user (7) are paired. Geolocation information, or Bluetooth proximity can be factored in to narrow the search between the fleet articles across the world. Bluetooth proximity can be assessed by looking at the available BT addresses nearby (including the one from the scooter), compare with a list of known BT addresses on file and enabling pairing only to a scooter that lives in that list. Alternatively, the scooter IMU signal or features extracted from it are encoded and advertised using Bluetooth or ultrasound or other RF method. The wearable picks up the advertising using the Bluetooth or the microphone or an RF transceiver respectively. If the features match, the scooter and the user are paired. This description refers to scooter ride sharing, but it can also apply to bicycle or other shared vehicles. Alternatively, communication between the article sensor (2) and the wearable (6) can be done using ultrasonic or infrared or RF communication protocols.

Another embodiment of the invention is to determine that two or more passengers are onboard a vehicle. In peer-to-peer ride sharing applications, where the identity of the driver needs to be confirmed, the motion profile of the driver's smartphone or wearable may be compared to the motion profile of the smartphone or wearable of a rider. This can be achieved by extracting features from each motion profile, communicating them to a pattern matching module that lives on a server, that determines the level of similarity of the two signals as well as the geolocation proximity of the driver and the rider. This is a case where pairing occurs between two users and not between user and article.

Another embodiment of the invention has an application in determining the operator/driver of a vehicle. Since the introduction of mobile technologies there has been a significant increase in road accidents that are attributed to the use of mobile phones while driving. The National Safety Council (NSC) analysis of National Highway Traffic Safety Administration (NHTSA) reported that fatal road accidents in United States attributed to smartphone usage while driving was 3,242 and 2,841 for 2017 and 2018, respectively. In this embodiment the steering wheel is equipped with an article sensor that captures the rotation rate and angle of the steering wheel using its IMU. The user is identified as the driver of the vehicle only if the rotation rate or angle profile captures by the IMU of a wearable (such as a smartwatch) is matching the rotation rate or angle of the article. By identifying the driver specific functionality of the car or other connected devices such as paired smart phones are enabled or disabled or otherwise limited. For instance, pairing between the steering wheel and the smartwatch can serve as an extra layer of security when trying to operate the vehicle. Moreover, in car sharing applications it can serve as an identification method between the car and the user that is just about to share the vehicle.

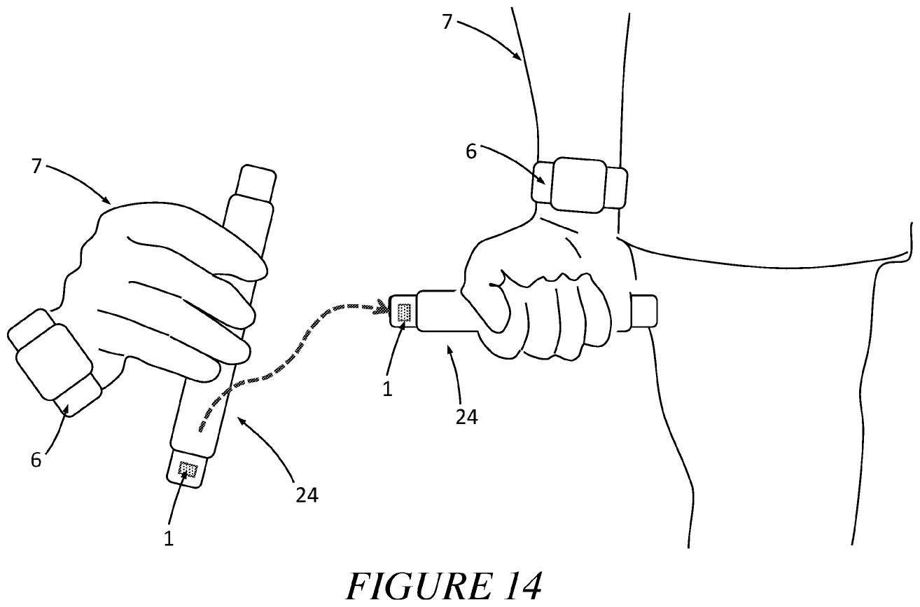

Another embodiment of the invention has an application in drug delivery, to confirm dose delivery. A sensor (1) can be placed on a drug container, sachet, blister-pack, pill-bottle, autoinjector (24), syringe, vial and capture the motion profile as showed in FIG. 14. When a user (7) is using the drug delivery device (24) or container that has a sensor (1) attached to it, the motion profile from the sensor (1) matches the motion profile from the user's wearable (6). If the similarity in these two profiles is high enough, the drug delivery or drug containing device is paired to the user (7). Pairing allows for drug adherence information to be extracted.

In one embodiment, the pairing occurs between an article and various parts of the supply chain including storage, handling and transportation. For example, a sensor can be placed on the packaging of a shipped good and then as the package goes through handling and transportation, the pairing occurs with each respective means. Pairing can occur with the delivery truck or the delivery person, by having sensors on the truck or the person respectively.

In another embodiment of the invention involves gun safety and adds and additional layer of safety for operating the said device. For example, a sensor can be mounted on the body of the gun and only if the motion pattern of the mounted article matches with the wearable of the authorized user—owner, the gun is paired to the user and can be unlocked and operated.

In one embodiment, the pairing occurs between an article and a buyer in a commercial store. The sensor is placed on the article and when the user wearing a wearable sensor takes it from the shelf, the motion patterns of the user and the article present similarity and thus the article is paired with the buyer. Alternatively, the shopping cart can be equipped with a sensor, and thus products that are in the cart will have a similar inertial signature. Once the user is paired to a cart, by means of pushing it and therefore matching his own inertial profile to that of the cart, the user can be also paired to all the contents of his cart. This method can be used for billing in teller-free retail stores or industrial or commercial warehouses.

The matching of the user and article motion profiles is done by means of algorithms that are designed to calculate features and optimize their number needed to classify tagged data. A list of feature selection algorithms includes, but is not limited to, Minimum redundancy feature selection (mRMR), a filter-based algorithm to select subset of features that maximize the mutual information while minimizing the redundancy of overlapping features (Relief, ReliefF and derivatives thereof), Gradient Boosting machines, tree-based algorithm that employs gradient descent for training and boosting method to improve weak classifiers (Gradient Boosting, XGBoost etc,), Support Vector Machines (SVM), Audio Search Algorithms (Combinatorial Hashing), Nearest Neighbors (KNN), Angular Metric for shape similarity (AMSS), Symbolic Aggregate Approximation (SAX) and other feature based methods. Feature selection is performed in order to minimize the amount of information needed to communicate between the user and the article sensors. Another class of methods to determine similarity (match) of the user and article motion profiles relies on recurrent neural networks, such as Long Short Term Memory (LSTM) or Attention Long Short Term Memory (ALSTM), or Multilayer Perceptron (MLP), or convolutional neural networks such as Fully Convolutional Neural Networks (FCN) or Ecostate Neural Networks (ESN) or other neural network based methods. The pattern matching algorithm classifies univariate or multivariate sensor data in rolling windows producing a similarity estimate.

The identification of activity based on the user and article motion profiles is done by means of algorithms that are designed to optimize the number of features needed to classify tagged data. A list of feature selection algorithms includes, but is not limited to, Minimum redundancy feature selection (mRMR), a filter-based algorithm to select subset of features that maximize the mutual information while minimizing the redundancy of overlapping features (Relief, ReliefF and derivatives thereof), Gradient Boosting machines, tree-based algorithm that employs gradient descent for training and boosting method to improve weak classifiers (Gradient Boosting, XGBoost etc,), Support Vector Machines (SVM) and others. The classification algorithm classifies univariate or multivariate sensor data in rolling windows producing an estimate of the activity. For example, given a training set, the classification algorithm can provide probability of one or the other exercise. In the case of a gym application, a similarity index can be used to examine how close the user motion profile is to the ideal motion profile as captured by elite athletes and provide feedback to the user.

In one embodiment, the identification of activity based on the user and article motion profiles is done by means of a convolutional neural networks (CNN) or recurrent neural network (RNN) or other deep learning neural networks, such as Gated Recurrent Unit (GRU), Long short-term memory (LSTM), Multilayer Perceptron (MLP) trained based on (tagged) data that takes convolutional input layers to generate a feature representation of the signal window vector that is trained by the recurrent layers. The identification can produce estimates of what the activity is. For example, given a training set, the algorithm can provide probability of one or the other exercise. In the case of a gym application, a similarity index can be used to examine how close the user motion profile is to the ideal motion profile as captured by elite athletes and provide feedback to the user.

In one embodiment, the matching between the sensor and the user motion profiles is done by means of frequency spectrum and phase extraction of a sliding window. Each of the two signals are analyzed using Fast Fourier Transform, or Discrete Fourier Transform or Discrete Cosine Transform or Discrete Wavelet Transform or Short-time Fourier Transform. A portion of the spectrum of each signal can be used to compare between the two motion profiles. The actual comparison can be done by identifying distance between the largest frequency peaks and by identifying the difference in their phase. A small difference means high similarity. In one embodiment, the cross correlation of the two transformed signals from the user and the article respectively can be used to find local maxima and therefore pair user to device. This method is similar to audio search algorithms (Avery Li-Chun Wang, 2003).

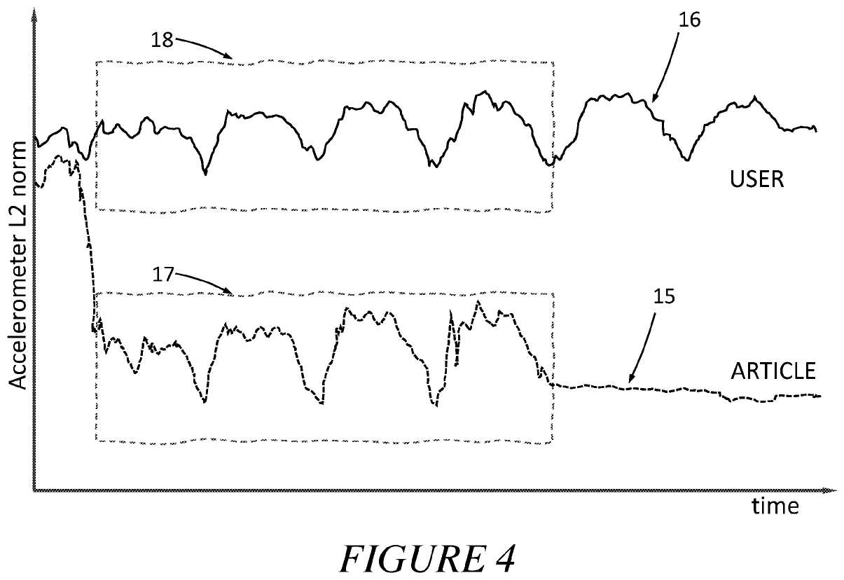

In one embodiment, two motion profiles, one coming from the wearable (16) and one from the article (15) present an area (17 and 18) where the similarity is high, signifying a potential interaction of the user with the article as shown in FIG. 4.

In one embodiment, the matching between the sensor and the user motion profiles is done by means of calculating the difference or the squared difference between two respective sliding windows of the signals.

In one embodiment, the matching between the sensor and the user motion profiles is done by means of calculating the area under the curve of the difference between the two signals.

In one embodiment, the matching between the sensor and the user motion profiles is done by means of comparing sets of univariate features: mean, variance, standard deviation, maximum, minimum, skewness, kurtosis, mean crossings, mean spectral energy, and a n-bin histograms. A built-in pretrained classification algorithm computes features in the wearable sensor or the article, respectively, and communicates them to the computing unit. The computing unit determines the similarity of the features using a similarity algorithm such as Dynamic time warping (DTW) between pairs of the article—sensor features, or the distance between the feature set/array belonging to the article sensor and the feature set/array belonging to the user sensor, or cross-correlation between the features belonging to the article sensor and determines. The similarity algorithm computes a score and if this score is above a threshold then pairing happens.

In one embodiment, the matching between the sensor and the user motion profiles is done by means of comparing sets of multivariate features including, but not limited to Dynamic time warping (DTW) between pairs of acceleration components, the weighted DTW with maximum of acceleration's band power, the phase difference calculated using the Hilbert transformation or similar between pairs of acceleration components, the spearman correlation coefficient between pairs of acceleration or rotation rate components, the cross-correlation between pairs of acceleration or rotation rate components, the power density of the cross-correlated signal between pairs of acceleration or rotation rate components at a specific band. The combination of multivariate features allows determining if a plurality of signals is co-occurring providing with a unique signature for the specific combination of signal pattern.

In one embodiment, the matching between the sensor and the user motion profiles is done by means of comparing features on the respective inertia time-frequency spectrograms. For the user motion data, a computer program can identify metrics in the time-frequency spectrum such as the number of peaks (N) or the dominant frequency or the sum of the dominant frequencies in the time-frequency spectrum. Let pᵢ denote each metric with 1=1 . . . N. The time associated with this metric is an epoch. For each pᵢ, a distance-map the M neighboring metrics is created. Let dᵢⱼ denote the matrix that contains these distances with j=1 . . . M and M<=N−1. A similar process is performed for the sensor motion data resulting in a matrix dᵢⱼ. Typically, d′ is smaller in the i-th dimension than d to save on computations. In one embodiment, d′ is simply a 1×M vector. The computer program then calculates the squared difference between each vector line of d′ from each vector line of d to find minima. If a minimum is found (for example when below a threshold) then the epoch that corresponds to that peak is considered to be a paired epoch. Pairing can happen retroactively via a server that hosts a decision-making algorithm, or real time by transmitting for example elements of vector line d′ using the Bluetooth advertising and comparing with the line vectors in d from a wearable.

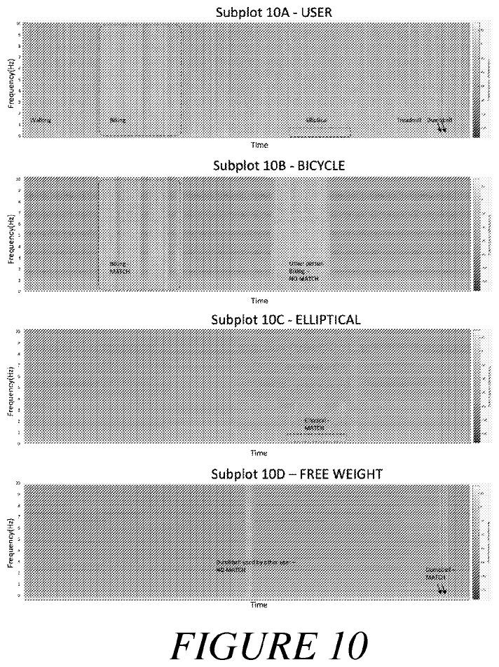

EXAMPLE: As shown in FIG. 10, time-frequency coming from several sources can be capture using a plurality of sensors that are attached to the user and several articles during an operating window. In this example, the user first rode a city bike from one location to another, then went to the gym where be rode the elliptical machine and then exercised using dumbbells. All the above mentioned articles are equipped each with a sensor that is capable of capturing and communicating the motion profile or features that are extracted from it. For this example, all the samples are synchronized in the time domain.

In one embodiment, the extracted features from the article sensor are encoded and transmitted in the advertising string of a Bluetooth communication module. The advertising string is picked up by the wearable Bluetooth sensor and the string is decoded to extract the features. A potential encoding scheme may involve the following formats:

Older Bluetooth versions: an up to 31 byte long (primary advertising) string that can include the necessary flags (type=0×01=flags), followed by a custom 128 bit unique identifier string (GUID) that contains encoded acceleration information and a custom local name used for reference and identification. The following table presents an example of advertising string, that contains a GUID that encodes motion profile information. In this example, the encoded motion profile information is created by taking the four last peaks of the L2 norm of the 3-axis accelerometer signal and calculating the time difference between them. These values are encoded in the Bluetooth advertising string so that other Bluetooth scanning devices can examine if pairing is to occur.

| Bluetooth | | | |
|---|---|---|---|
| advertising | | | 0908 |
| string: | 0201 06 | 1107 04CF 058D 03AA 0462 | 53464F31393737 |
| Explanation: | Flags | Vector containing time | Name of device |
| | | difference between 4 last | |
| | | consecutive major | Value: |
| | | accelerometer peaks in ms | “XSFO1977” |
| | | Value: 1231-1421-0938-1122 | |

The Bluetooth advertising is normally silent to save battery, and is awaken by an inertial change when detected by the IMU: in that case the Bluetooth starts transmitting advertising packages at high rate. If a registered user is in the proximity, the advertising is picked up by the wearable. The wearable looks for devices that have an identifier string that matches a preloaded lookup table (in this example “SF01977”). It then looks at the vector containing the 4 last consecutive major accelerometer peaks time difference in milliseconds. The wearable probes its IMU for the last 4+n consecutive accelerometer peaks in milliseconds:

| | | | | | | | | |
|---|---|---|---|---|---|---|---|---|
| Wearable (user) peaks difference | 0023 | 2422 | 9423 | 1240 | 1529 | 1001 | 1124 | 1543 |
| Article peaks difference kernel | 1231 | 1421 | 0938 | 1122 | | | | |
| Sq Difference between vectors | 8629 | 8106 | 8216 | 125 | 690 | | | |
| (convolution result) | | | | | | | | |

The square root of the squared difference between the two vectors as calculated using a sliding window presents a minimum (=125) in position 4 so a close match has been identified. In that case wearable (6) and hence, user (7) is paired to article (1).

Newer Bluetooth versions: the auxiliary advertising channels can be used offering up to 256 bytes of string. Other motion features can also be transmitted using these channels such as spectrum or other statistical identifying information.

In one embodiment, the extracted features from the article sensor are encoded and transmitted using ultrasound. They are received by a wearable, or a hub and communicated to the computing unit.

In one embodiment, the extracted features from the article sensor are encoded and transmitted wirelessly to a wireless hub which then transmits to the cloud. Similarly, the extracted features from the user sensor are encoded and transmitted wirelessly to a wireless hub which then transmits to the cloud and communicated to the computing unit.

1-57. (canceled)

58. A method for pairing a user to an article, comprising:
providing a plurality of users, each of the plurality of users having a respective wearable device associated therewith, each wearable device or each of the plurality of users including one or more user sensors configured to capture a motion profile of the user that is wearing the wearable device, each wearable device configured to communicate the motion profile of each respective user to a matching module;
providing a plurality of articles, each of the plurality of articles having at least one article sensor associated therewith, the at least one article sensor having an inertial moment unit, a processing unit, and a communications unit, wherein the at least one article sensor is configured to sense and determine a motion profile of the article and transmit information indicative of the motion to the matching module;
transmitting the information indicative of the motion profile of the user to the matching module;
transmitting the information indicative of the motion profile of the article to the matching module;
receiving at the matching module a plurality of user motion profiles for each of the plurality of users;
concurrently with receiving the plurality of user motion profiles, receiving at the matching module a plurality of article motion profiles for each of the plurality of articles;
using the matching module to compare the plurality of user motion profiles with the plurality of article matching profiles; and
determining at the matching module that one user of the plurality of users is spatially interacting with one article of the plurality of articles when the respective user motion profile corresponding to the one user substantially matches with the respective article motion profile corresponding to the one article.

59. The method of claim 58, further comprising acquiring sensor signals from the at least one sensor on each of the plurality of articles, the sensor signals being indicative of a change in at least one of inertia, force, stress, and strain that the at least one article is subjected to, and/or capacitance, resistance or a voltage profile of an electrical property of the at least one article.

60. The method of claim 58, wherein determining that the one user is spatially interacting with the one article further comprises transmitting a matching decision to the wearable device of the one user.

61. The method of claim 59, further comprising wirelessly pairing the wearable device of the one user with the one article without user interaction with the wearable device.

62. The method of claim 61, wherein wirelessly pairing occurs when and while the one user physically interacts with at least a portion of the one article.

63. The method of claim 58, wherein the one article is one of a sports equipment item, a mobile phone, a computer tablet, a computer stylus, a toy, a building door handle, an appliance door handle, a utensil, a drug delivery device, a transportation vehicle, a chair, a seat in a vehicle, a digital scale, a bed, or a bed mattress, and wherein at least one of the plurality of wearable devices is one of a smart watch, a smart ring, a smart phone, and a smart patch.

64. The method of claim 58, wherein transmitting the information indicative of the user motion profile or the article motion profile is accomplished using wireless communication protocols, the wireless communication protocols comprising at least one of Bluetooth, wifi, ultrasound, Ultra-Wideband, radio frequency, and optical communication.

65. The method of claim 58, wherein the plurality of users includes a shipper of goods, and wherein the plurality of articles includes a shipped good.

66. The method of claim 65, wherein the shipper of goods is a delivery vehicle or a delivery person operating the delivery vehicle.

67. The method of claim 58, wherein the plurality of users includes a retail customer visiting a store, and wherein the plurality of articles includes items for sale in the store.

68. The method of claim 58, wherein sensing and determining the motion profile of the user includes extracting user features, wherein sensing and determining the motion profile of the article includes extracting article features, and wherein comparing the plurality of user motion profiles with the plurality of article matching profiles is accomplished by comparing the user features with the article features.

69. The method of claim 68, wherein the pattern module compares the user features and the article features by at least one of:
convolution of the user and article features and finding maxima in a result of the convolution;
cross-correlating the user and article features and finding maxima in a result of the cross-correlation; and
performing a Short-Fourier transform or Fast Fourier Transform or Discrete Cosine Transform on profile streams of each of the user and article features and finding minima in a result of the transform or comparing a temporal distance between local peaks in the profile streams;

70. A system for digitally pairing a user to an article, comprising:
at least one wearable device adapted to be associated with a user, the at least one wearable device including one or more user sensors configured to capture a motion profile of the user that is wearing the wearable device;
at least one article, the at least one article having at least one article sensor associated therewith, the at least one article sensor having an inertial moment unit, a processing unit, and a communications unit, wherein the at least one article sensor is configured to sense and determine a motion profile of the at least one article.
a matching module configured to receive information indicative of the motion profile of the at least one user concurrently with the motion profile of the at least one article, wherein the matching module is configured to compare the plurality of user motion profiles with the plurality of article matching profiles, and to determine that the user is spatially interacting with the article when the user motion profile substantially matches with the article motion profile.

71. The system of claim 70, further comprising a plurality of users and a plurality of articles, wherein the matching module is further configured to identify the user among the plurality of users and the article among the plurality of articles.

72. The system of claim 71, wherein the matching module is further configured to concurrently receive a plurality of user motion profiles and a plurality of article motion profiles.

73. The system of claim 70, wherein the at least one sensor is configured to provide sensor signals to the matching module that are indicative of a change in at least one of inertia, force, stress, and strain that the at least one article is subjected to.

74. The system of claim 70, wherein determining that the one user is spatially interacting with the at least one article in the matching module further comprises transmitting a matching decision to the at least one wearable device.

75. The system of claim 70, wherein the at least one wearable device is further configured to wirelessly pair with the at least one article sensor upon the determination that the user is spatially interacting with the article.

76. The system of claim 75, wherein the at least one wearable device is further configured to unpair with the at least one article sensor upon a determination at the matching module that the user is no longer spatially interacting with the article.
